(19) United States
(12) Patent Application Publication
(10) Pub. No.: US 2011/0300088 A1
(43) Pub. Date: Dec. 8, 2011

(54) WATER-INSOLUBLE REACTION PRODUCT OF A POLYAMINE AND AN OIL-SOLUBLE POLAR MODIFIED POLYMER

(75) Inventors: Hy Si Bui, Piscataway, NJ (US); Mohamed Kanji, Edison, NJ (US); Nghi Van Nguyen, Edison, NJ (US); Chunhua Li, Scotch Plains, NJ (US); Anita Chon Tong, Garwood, NJ (US); Susan Halpern, Paramus, NJ (US); Bruno Bavouzet, Gentilly (FR); Anil Shah, East Windsor, NJ (US); Ana Kljuic, New York, NY (US)

(73) Assignee: L'OREAL S.A., Paris (FR)

(21) Appl. No.: 13/132,811

(22) PCT Filed: Dec. 9, 2009
(86) PCT No.: PCT/US09/67339

§ 371 Date: Jul. 29, 2011

Related U.S. Application Data
(60) Provisional application No. 61/120,970, filed on Dec. 9, 2008.
(60) Provisional application No. 61/221,379, filed on Jun. 29, 2009.

(51) Int. Cl. A61K 8/92; A61Q 1/06; A61Q 1/02; A61Q 1/10; C08G 73/04; A61K 47/34

(52) U.S. Cl. 424/64; 528/342; 424/63; 514/772.7

(57) ABSTRACT

The invention relates to a water-insoluble reaction product of an oil-soluble polar modified polymer and a polyamine.

### FIELD OF THE INVENTION

The present invention relates to a water-insoluble reaction product of at least one polyamine compound having at least two amine groups and at least one oil-soluble polar modified polymer. The water-insoluble reaction product can act as a carrier or matrix for desired agents. Such reaction products have industrial, pharmacological and/or cosmetic applicability.

### DISCUSSION OF THE BACKGROUND

U.S. Pat. No. 6,492,455 discloses water-soluble reaction products of polyamines and C6 olefin/maleic anhydride copolymers. Because these compositions are water-soluble, addition of water to such reaction products renders the products unsuitable for applications requiring water-insolubility. For example, such reaction products are unsuitable for use as a solid carrier containing colorant (for example, industrial pigments) or active agents (for example, pharmaceuticals) because the reaction product breaks down upon exposure to water.

Thus, there remains a need for improved water-insoluble products which can function as a carrier and/or matrix for desired agents.

### SUMMARY OF THE INVENTION

The present invention relates to water-insoluble reaction products of at least one polyamine compound having at least two amine groups and at least one oil-soluble polar modified polymer.

The present invention also relates to compositions comprising (1) a water-insoluble reaction product of at least one polyamine compound having at least two amine groups and at least one oil-soluble polar modified polymer; and (2) a desired agent such as a colorant or pharmacologically active agent.

The present invention also relates to solid compositions comprising (1) a water-insoluble reaction product of at least one polyamine compound having at least two amine groups and at least one oil-soluble polar modified polymer; and (2) water. Preferably, such compositions further comprise a desired agent.

The present invention further relates to cosmetic compositions comprising a water-insoluble reaction product of at least one polyamine compound having at least two amine groups and at least one oil-soluble polar modified polymer.

The present invention also relates to methods of treating, caring for and/or making up keratinous material (for example, skin, eyes, eyelashes or lips) by applying cosmetic compositions of the present invention to the keratinous material in an amount sufficient to treat, care for and/or make up the keratinous material.

The present invention also relates to methods of improving the feel or texture properties of a cosmetic composition upon application to a keratin material, and/or the adhesion, long-wear and/or transfer-resistance properties of a cosmetic composition, comprising adding to a composition a water-insoluble reaction product of at least one polyamine compound having at least two amine groups and at least one oil-soluble polar modified polymer.

The present invention also relates to methods of making a water-insoluble reaction product by reacting a polyamine compound having at least two amine groups and an oil-soluble polar modified polymer to form the water-insoluble reaction product.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc. In a preferred embodiment of the present invention, little or no composition is transferred from the wearer.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Water-insoluble,” as used herein, means that the reaction product retains its structure and does not solubilize upon exposure to water.

The compositions and methods of the present invention can comprise, consist of, or consist essentially of the essential elements and limitations of the invention described herein, as well as any additional or optional ingredients, components, or limitations described herein or otherwise useful.

In accordance with the present invention, the “hardness” of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf, including all ranges and subranges therebetween.

This hardness is measured in one of two ways. A first test for hardness is according to a method of penetrating a probe into the composition and in particular using a texture analyzer (for example TA-XT21 from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within preferred embodiments of the invention.

Oil-Soluble Polar Modified Polymer

According to the present invention, compositions comprising at least one oil-soluble polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil.

Suitable monomers for the hydrophobic homopolymers and/or copolymers include, but are not limited to, cyclic, linear or branched, substituted or unsubstituted, C2-C20 compounds such as, for example, styrene, ethylene, propylene, isopropylene, butylene, isobutylene, pentene, isopentene, isoprene, hexene, isohexene, decene, isodecene, and octadecene, including all ranges and subranges therebetween. Preferably, the monomers are C2-C8 compounds, more preferably C2-C6 compounds, and most preferably C2-C4 compounds such as ethylene, propylene and butylene.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to the present invention, the polar modified polymer is oil-soluble: that is, the polymer does not contain a sufficient amount of hydrophilic unit(s) to render the entire polymer water-soluble or oil-insoluble. According to preferred embodiments, the polar modified polymer contains the same amount of hydrophobic monomer as hydrophilic unit (1:1 ratio) or more hydrophobic monomer than hydrophilic unit. According to particularly preferred embodiments, the polar modified polymer contains 50% or less hydrophilic unit(s) (based on weight of the polymer), 40% or less hydrophilic unit(s), 30% or less hydrophilic unit(s), 20% or less hydrophilic unit(s), 10% or less hydrophilic unit(s), 5% or less hydrophilic unit(s), 4% or less hydrophilic unit(s), or 3% or less hydrophilic unit(s).

Preferably, the polar modified polymer has from about 0.5% to about 10% hydrophilic units, more preferably from about 1% to about 8% hydrophilic units by weight with respect to the weight of the polymer, including all ranges and subranges therebetween. Particularly preferred hydrophilically modified polymers are ethylene and/or propylene homopolymers and copolymers which have been modified with maleic anhydride units.

According to preferred embodiments of the present invention, the polar modified polymer is a wax. According to particularly preferred embodiments, the polar modified wax is made via metallocene catalysis, and includes polar groups or units as well as a hydrophobic backbone. Suitable modified waxes include those disclosed in U.S. patent application publication no. 20070031361, the entire contents of which is hereby incorporated by reference. Particularly preferred polar modified waxes are C2-C3 polar modified waxes.

In accordance with preferred embodiments of the present invention, the polar modified wax is based upon a homopolymer and/or copolymer wax of hydrophobic monomers and has a weight-average molecular weight Mw of less than or equal to 25 000 g/mol, preferably of 1000 to 22 000 g/mol and particularly preferably of 4000 to 20,000 g/mol, a number-average molecular weight Mn of less than or equal to 15 000 g/mol, preferably of 500 to 12 000 g/mol and particularly preferably of 1000 to 5000 g/mol, a molar mass distribution Mw/Mn in the range from 1.5 to 10, preferably from 1.5 to 5, particularly preferably from 1.5 to 3 and especially preferably from 2 to 2.5, which have been obtained by metallocene catalysis. Also, the polar modified wax preferably has a melting point above 75° C., more preferably above 90° C. such as, for example, a melting point between 90° C. and 160° C., preferably between 100° C. and 150° C., including all ranges and subranges therebetween.

In the case of a copolymer wax, it is preferable to have, based on the total weight of the copolymer backbone, 0.1 to 30% by weight of structural units originating from the one monomer and 70.0 to 99.9% by weight of structural units originating from the other monomer. Such homopolymer and copolymer waxes can be made, for example, by the process described in EP 571 882, the entire contents of which is hereby incorporated by reference, using the metallocene catalysts specified therein. Suitable preparation processes include, for example, suspension polymerization, solution polymerization and gas-phase polymerization of olefins in the presence of metallocene catalysts, with polymerization in the monomers also being possible.

Polar modified waxes can be produced in a known manner from the hompopolymers and copolymers described above by oxidation with oxygen-containing gases, for example air, or by graft reaction with polar monomers, for example maleic acid or acrylic acid or derivatives of these acids. The polar modification of metallocene polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable polar modified waxes include, but are not limited to, homopolymers and/or copolymers of ethylene and/or propylene groups which have been modified with hydrophilic units such as, for example, maleic anhydride, acrylate, methacrylate, polyvinylpyrrolidone (PVP), etc. Preferably, the C₂-C₃ wax has from about 0.5% to about 10% hydrophilic units, more preferably from about 1% to about 8% hydrophilic units by weight with respect to the weight of the wax, including all ranges and subranges therebetween. Particularly preferred hydrophilically modified waxes are ethylene and/or propylene homopolymers and copolymers which have been modified with maleic anhydride units.

Particularly preferred C₂-C₃ polar modified waxes for use in the present invention are polypropylene and/or polyethylene-maleic anhydride modified waxes (“PEMA,” “PPMA.” “PEPPMA”) commercially available from Clariant under the trade name LICOCARE or LICOCENE, Specific examples of such waxes include products marketed by Clariant under the LicoCare name having designations such as PP207.

Other suitable polar modified polymers include, but are not limited to A-C 573 A (ETHYLENE-MALEIC ANHYDRIDE COPOLYMER; prop Point, Mettler: 106° C.) from Honeywell, A-C 596 A (PROPYLENE-MALEIC ANHYDRIDE COPOLYMER; prop Point, Mettler: 143° C.) from Honeywell, A-C 597 (PROPYLENE-MALEIC ANHYDRIDE COPOLYMER; prop Point, Mettler: 141° C.) from Honeywell, ZeMac® copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly(maleic anhydride-octadecene) sold by Chevron Philips Chemcial Co., poly (ethylene-co-butyl acrylate-co-maleic anhydride) sold under the trade name of Lotader (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the Lotader name, and isobutylene maleic anhydride copolymer sold under the name ACO-5013 by ISP.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orientation (for example, atactic, isotactic or syndiotactic along the backbone).

Preferably, the polar modified polymer(s) represent from about 1% to about 30% of the total weight of the composition, more preferably from about 3% to about 20% of the total weight of the composition, and most preferably from about 5% to about 15%, including all ranges and subranges therebetween.

Polyamine Compound

According to the present invention, compositions comprising at least one polyamine compound are provided. In accordance with the present invention, the polyamine compound has at least two primary amine groups available to react with hydrophilic groups of the oil-soluble polar modified polymer.

According to particularly preferred embodiments, the polyamine compound is a polyalkyleneimine, preferably a C2-C5 polyalkyleneamine compound, more preferably a polyethyleneimine or polypropyleneimine. Most preferably, the polyalkylenamine is polyethyleneimine (“PEI”). The polyalkyleneamine compound preferably has an average molecular weight range of from 500-200,000, including all ranges and subranges therebetween.

According to preferred embodiments, compositions of the present invention contain polyethyleneimine compounds in the form of branched polymers. Commercially available examples of such polymers are available from BASF under the tradename LUPASOL or POLYIMIN. Non-limiting examples of such polyethyleneimines include Lupasol® PS, Lupasol® PL, Lupasol® PR8515, Lupasol® G20, Lupasol® G35.

According to other embodiments of the present invention, polyamines such as polyethyleneimines and polypropyleneimines can be in the form of dendrimers. Non-limiting examples of such dendrimers are manufactured by the company DSM, and/or are disclosed in U.S. Pat. No. 5,530,092 and U.S. Pat. No. 5,610,268, the contents of which are hereby incorporated by reference. Commercially available examples of such polymers include polyamidoamine or polypropyleneimine polymers from DENDRITECH sold under the STARBURST® name.

According to other embodiments of the present invention, derivatives of polyalkyleneamines are suitable polyamines. Such derivatives include, but are not limited to, alkylated derivatives, the addition products of alkylcarboxylic acids to polyalkyleneamines, the addition products of ketones and of aldehydes to polyalkyleneamines, the addition products of isocyanates and of isothiocyanates to polyalkyleneamines, the addition products of alkylene oxide or of polyalkylene oxide block polymers to polyalkyleneamines, quaternized derivatives of polyalkyleneamines, the addition products of a silicone to polyalkyleneamines, and copolymers of dicarboxylic acid and polyalkyleneamines. Even further suitable polymamines include, but are not limited to, polyvinylimidazoles (homopolymers or copolymers), polyvinylpyridines (homopolymers or copolymers), compounds comprising vinylimidazole monomers (see, for example, U.S. Pat. No. 5,677,384, hereby incorporated by reference), and polymers based on amino acids containing a basic side chain (preferably selected from proteins and peptides comprising at least 5%, preferably at least 10% of amino acids selected from histidine, lysine and arginine). Such suitable polyamines as described above include those disclosed and described in U.S. Pat. No. 6,162,448, the contents of which are hereby incorporated by reference. Commercially available examples of such polymers include polyvinylamine/formamide such as those sold under the Lupamine® name by BASF, chitosan from vegetable origin such as those sold under the Kiosmetine® or Kitozyme® names, or copolymer 845 sold by ISP.

According to preferred embodiments, the at least one polyamine compound is present in the composition of the present invention in an amount ranging from about 0.1 to about 10% by weight, more preferably from about 0.2 to about 5% by weight, based on the total weight of the composition, including all ranges and subranges within these ranges.

Preferably, the amount of polyamine compound reacted with the oil-soluble polar modified polymer is such that at least two amine groups on the polyamine compound react with the oil-soluble polar modified polymer to form links or bonds between the amine groups and the hydrophilic groups of the oil-soluble polar modified polymer. The appropriate amount of polyamine compound to react with the oil-soluble polar modified polymer to obtain a reaction product can be easily determined, taking into account the number/amount of reactive amine groups on the polyamine compound and the number/amount of corresponding reactive groups on the oil-soluble polar modified polymer (for example, maleic anhydride groups). According to preferred embodiments, excess oil-soluble polar modified polymer (as determined by the relative number/amount of corresponding reactive groups on the polymer as compared to the reactive amine groups on the polyamine) is reacted with polyamine. Preferably, the polyamine to oil-soluble polar modified ratio is between 0.005 and 1, preferably between 0.006 and 0.5, and preferably between 0.007 and 0.1, including all ranges and subranges therebetween.

Reaction Product

According to the present invention, the oil-soluble polar modified polymer is reacted with the polyamine compound to form a reaction product of an oil-soluble polar modified polymer and a polyamine. In accordance with the present invention, the reaction product is water-insoluble.

Preferably, when the reaction product is exposed to water, water can be incorporated within the reaction product. Thus, rather than forming an aqueous solution when exposed to water, the reaction product preferably maintains its structure. Preferably, the reaction product forms a matrix or carrier containing water. According to preferred embodiments of the present invention, water comprising a desired agent can be incorporated into the reaction product such that the reaction product is a matrix or carrier for the water and/or desired agent.

According to the present invention, any suitable amine chemistry can be used to form the reaction product of the present invention. The exact chemistry will depend upon the nature of the corresponding reactive group of the oil-soluble polar modified polymer hydrophilic group with which the at least two amine groups of the polyamine will react. However, once the nature of the corresponding reactive groups is known, their reaction with the at least two amine groups of the polyamine will proceed according to known amine chemistry principles.

According to preferred embodiments, the oil-soluble polar modified polymer is in an oil carrier, and the polyamine compound is in an aqueous carrier, and the reaction occurs by combining the oil carrier and the aqueous carrier. Because the oil-soluble polar modified polymer is typically solid at room temperature, the oil carrier is preferably heated to liquefy the polymer prior to combination with the aqueous carrier. Preferably, the oil carrier is heated beyond the melting point of the oil-soluble polar modified polymer, typically up to about 80° C., 90° C. or 100° C. Although not wanting to be bound by any particular theory, it is believed that at a temperature below 100° C., the reaction of oil-soluble polar modified polymer with the primary amine group of the polyamine opens the anhydride ring to form a half acid and half amide crosslinked product. However, at a temperature above 100° C., the reaction of oil-soluble polar modified polymer with the primary amine group of the polyamine opens the anhydride ring to form an imide crosslinked product. The former product is preferred over the latter product. It is not necessary for all amine groups and all hydrophilic groups to react with each other to form the reaction product. Rather, it is possible that the composition may contain free polyamine and/or free oil-soluble polar modified polymer in addition to the reaction product.

Although not wanting to be bound by any particular theory, it is also believed that the polyamine(s) can be non-covalently assembled with the polar modified polymer(s) by electrostatic interaction between an amine group of the polyamine and a hydrophilic group (for example, carboxylic acid group associated with maleic anhydride groups) of the polar modified polymer to form a supramolecule. For example, with specific reference to maleic anhydride groups, in the presence of water these groups can open to form dicarboxylic acid groups which can interact with protonated primary amines of the polyamine through ionic interaction to form a polymer-polymer complex with hydrophilic core crosslinkers and a hydrophobic network that act as supramolecular capsule. If a large amount of maleic anhydride groups are present, the secondary amine groups of polyamine are also protonated and interact with alkyl carboxylates.

According to preferred embodiments, the oil carrier comprises volatile and/or non-volatile oils. Such oils can be any acceptable oil including but not limited to silicone oils and/or hydrocarbon oils.

According to preferred embodiments, the oil carrier comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other preferred embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to preferred embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments of the present invention, the oil carrier, the aqueous carrier, or both comprise a desired agent to be incorporated within the reaction product. The desired agent can be, for example, any colorant (pigment, dye, etc.), any pharmaceutically or cosmetically active agent, or any film forming agent known in the art. Such a desired agent can be incorporated into the reaction product of the present invention and can be active during subsequent use of a composition comprising the reaction product. For example, a cosmetic makeup composition or a paint composition comprising colorant within the reaction product can provide colorant and/or film forming agent to a substrate (skin, lips, wall, frame, etc.) during use to provide the substrate with the desired film and/or color. Similarly, a pharmaceutical or cosmetic composition comprising a pharmaceutically active agent can provide such active agent to the patient or consumer upon use (for example, a transdermal patch containing the reaction product of the present invention within which is a pharmaceutically or cosmetically active agent, or a tablet or capsule containing the same reaction product/active agent combination).

Acceptable colorants include pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow.

Representative nacreous pigments include white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative pigments include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium,

Acceptable film forming agents and/or rheological agents are known in the art and include, but are not limited to, those disclosed in U.S. patent application publication no. 2004/0170586, the entire contents of which is hereby incorporated by reference.

Non-limiting representative examples of acceptable film forming/rheolgocial agents include silicone resins such as, for example, MQ resins (for example, trimethylsiloxysilicates), T-propyl silsesquioxanes and MK resins (for example, polymethylsilsesquioxanes), silicone esters such as those disclosed in U.S. Pat. Nos. 6,045,782, 5,334,737, and 4,725,658, the disclosures of which are hereby incorporated by reference, polymers comprising a backbone chosen from vinyl polymers, methacrylic polymers, and acrylic polymers and at least one chain chosen from pendant siloxane groups and pendant fluorochemical groups such as those disclosed in U.S. Pat. Nos. 5,209,924, 4,693,935, 4,981,903, 4,981,902, and 4,972,037, and WO 01/32737, the disclosures of which are hereby incorporated by reference, polymers such as those described in U.S. Pat. No. 5,468,477, the disclosure of which is hereby incorporated by reference (a non-limiting example of such polymers is poly(dimethylsiloxane)-g-poly(isobutyl methacrylate), which is commercially available from 3M Company under the tradename VS 70 IBM).

Suitable examples of acceptable liposoluble polymers include, but are not limited to, polyalkylenes, polyvinylpyrrolidone (PVP) or vinylpyrrolidone (VP) homopolymers or copolymers, copolymers of a C₂ to C₃₀, such as C₃ to C₂₂ alkene, and combinations thereof. As specific examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

One type of block copolymer which may be employed in the compositions of the present invention is a thermoplastic elastomer. The hard segments of the thermoplastic elastomer typically comprise vinyl monomers in varying amounts. Examples of suitable vinyl monomers include, but are not limited to, styrene, methacrylate, acrylate, vinyl ester, vinyl ether, vinyl acetate, and the like.

The soft segments of the thermoplastic elastomer typically comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention include block copolymers e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In preferred embodiments, the thermoplastic elastomer of the present invention may be chosen from the class of Kraton™ rubbers (Shell Chemical Company) or from similar thermoplastic elastomers. Kraton™ rubbers are thermoplastic elastomers in which the polymer chains comprise a di-block, tri-block, multi-block or radial or star block configuration or numerous mixtures thereof. The Kraton™ tri-block rubbers have polystyrene (hard) segments on each end of a rubber (soft) segment, while the Kraton™ di-block rubbers have a polystyrene (hard) segment attached to a rubber (soft) segment. The Kraton™ radial or star configuration may be a four-point or other multipoint star made of rubber with a polystyrene segment attached to each end of a rubber segment. The configuration of each of the Kraton™ rubbers forms separate polystyrene and rubber domains.

Each molecule of Kraton™ rubber is said to comprise block segments of styrene monomer units and rubber monomer and/or co-monomer units. The most common structure for the Kraton™ triblock copolymer is the linear A-B-A block type styrene-butadiene-styrene, styrene-isoprene-styrene, styrene-ethylenepropylene-styrene, or styrene-ethylenebutylene-styrene. The Kraton™ di-block is preferably the AB block type such as styrene-ethylenepropylene, styrene-ethylenebutylene, styrene-butadiene, or styrene-isoprene. The Kraton™ rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by Exxon Dow under the tradename Vector™.

Other thermoplastic elastomers useful in the present invention include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

Acceptable film forming/rheological agents also include water soluble polymers such as, for example, high molecular weight crosslinked homopolymers of acrylic acid, and Acrylates/C10-30 Alkyl Acrylate Crosspolymer, such as the Carbopol and Pemulen®; anionic acrylate polymers such as Salcare® AST and cationic acrylate polymers such as Salcare® SC96; acrylamidopropylttrimonium chloride/acrylamide; hydroxyethyl methacrylate polymers, Steareth-10 Allyl Ether/Acrylate Copolymer; Acrylates/Beheneth-25 Metacrylate Copolymer, known as Aculyn® 28; glyceryl polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof.

According to preferred embodiments of the present invention, compositions comprising the reaction product of the present invention can comprise substantial amounts of water. Preferably, compositions of the present invention comprise from about 5% to about 50% water, more preferably from about 15% to about 45% water, and more preferably from about 25% to about 40% water by weight with respect to the total weight of the composition, including all ranges and subranges therebetween. According to particularly preferred embodiments, compositions comprising the reaction product of the present invention and at least 25% water are solid compositions. Such solid compositions are preferably in the form of a stick (for example, a lipstick or a stick foundation).

Compositions comprising the reaction product of the present invention can optionally further comprise any additive usually used in the field(s) under consideration. For example, dispersants such as poly(12-hydroxystearic acid), antioxidants, essential oils, sunscreens, preserving agents, fragrances, fillers, neutralizing agents, cosmetic and dermatological active agents such as, for example, emollients, moisturizers, vitamins, essential fatty acids, surfactants, silicone elastomers, pasty compounds, viscosity increasing agents such as waxes or liposoluble/lipodispersible polymers, and mixtures thereof can be added. A non-exhaustive listing of such ingredients can be found in U.S. patent application publication no. 2004/0170586, the entire contents of which are hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

In one embodiment of the present invention, the compositions of the present invention are substantially free of silicone oils (i.e., contain less than about 0.5% silicone oils). In another embodiment, the compositions are substantially free of non-silicone oils (i.e., contain less than about 0.5% non-silicone oils). In another embodiment, the compositions are substantially free of non-volatile oils (i.e., contain less than about 0.5% non-volatile oils).

One particularly preferred embodiment of the present invention is an emulsion which is substantially free of surfactant (that is, less than 3% of surfactant), essentially free of surfactant (that is, less than 2% surfactant), or free of surfactant (that is, less than 0.5% surfactant).

Another particularly preferred embodiment of the present invention is a composition which contains so little elastomer that the presence of such elastomer not affect the cosmetic properties of the composition. Preferably, the compositions are substantially free of such elastomers (i.e., contain less than about 0.5% elastomer), essentially free of such elastomers (i.e., contain less than about 0.25% elastomer) or free of such elastomer (i.e., contain no elastomer).

According to other embodiments of the present invention, the compositions of the present invention are anhydrous. By “anhydrous,” it is meant that the composition contains substantially no water (that is, less than about 5% by weight of the composition of water).

According to other preferred embodiments, methods of treating, caring for and/or enhancing the appearance of keratinous material by applying compositions of the present invention to the keratinous material in an amount sufficient to treat, care for and/or enhance the appearance of the keratinous material are provided. In accordance with these preceding preferred embodiments, the compositions of the present invention comprising at least one oil-soluble polar modified polymer and at least one polyamine compound are applied topically to the desired area of the keratin material in an amount sufficient to treat, care for and/or enhance the appearance of the keratinous material. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, improved waterproof characteristics, improved feel upon application (for example, texture, reduced drag or tackiness), increased anti-smudging properties, shine/color characteristics and/or increased long wear properties are provided.

According to other embodiments of the present invention, methods of improving the anti-smudging, waterproof, transfer-resistance and/or long wear properties of a composition, comprising adding at least one oil-soluble polar modified polymer and at least one polyamine to the composition are provided. In accordance with this embodiment, the at least one oil-soluble polar modified polymer and the at least one polyamine are present in amounts sufficient to achieve the desired result.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Examples 1-4

| Component | EX 1 | EX 2 | EX 3 | EX 4 |
|---|---|---|---|---|
| ISOHEXADECANE | 83 | 81 | 79 | 75 |
| PP207 (*) | 15 | 15 | 15 | 15 |
| Lupasol G 35 PEI | 2 | 4 | 6 | 10 |
| (PolyEthyleneImine) | | | | |
| (50% SOLID/50% WATER) | | | | |
| Total | 100 | 100 | 100 | 100 |

(*) PP207 is a linear polypropylene-ethylene-maleic anhydride copolymer wax commercially available from Clariant under the tradename LICOCARE PP207 LP 3349.

Procedure
In container A, PP207 was melted in the isohexadecane until fully dissolved. The temperature was brought to 90° C.
Added LUPASOL G 35 PEI (PolyEthylenelmine) to the container A dropwise with a pipet at high shear (˜700 rpm);
Heat was maintained at 70° C.-80° C. for 20 minutes while maintaining high shear mixing;
The mixture was cooled to room temperature while mixing.
The final products of examples 1-4 were soft gels, which were insoluble in water.

### Example 5-7

| Component | EX 5 | EX 6 | EX 7 |
|---|---|---|---|
| ISOHEXADECANE | 75 | 65 | 55 |
| PP207 (*) | 15 | 15 | 15 |
| Lupasol G 35 PEI | 10 | 10 | 10 |
| (PolyEthyleneImine) | | | |
| (50% SOLID/50% WATER) | | | |
| WATER | 0 | 10 | 25 |
| Total | 100 | 100 | 100 |

(*) PP207 is a linear polypropylene-ethylene-maleic anhydride copolymer wax commercially available from Clariant under the tradename LICOCARE PP207 LP 3349.

Procedure

In container A, PP207 was melted in the isohexadecane until fully dissolved. The temperature was brought to 90° C.
In separate container B, LUPASOL G 35 PEI (PolyEthylenelmine) and water were mixed at room temperature;
B was added to A dropwise with a pipet at high sheer (˜700 rpm);
Heat was maintained at 70° C.-80° C. for 20 minutes while maintaining high shear mixing;
The mixture was cooled to room temperature while mixing.
The final products of examples 5-7 were gels, which could entrap high amounts of water but were insoluble in water.

### Example 8

Gel material for a cosmetic formulation

| Phase | Component | % wt |
|---|---|---|
| A | PP207 (*)) | 14.45% |
| A | Isododecane | 41.54% |
| B | Lupasol G 35 PEI | 2.49% |
| | (PolyEthyleneImine) | |
| | (50% SOLID/50% WATER) | |
| B | Potassium Cetyl Phsphate | 4.15% |
| B | DI Water | 36.54% |
| C | Phenoxyethanol | 0.83% |
| Total | | 100% |

### Procedure of Making Example 8

1. In the main beaker A, Isododecane, Polypropylene-ethylene-Maleic Anhydride Copolymer wax (PP207) were added, The contents were then heated to 90° C. until all solids melted.
2. In another beaker B, added deionized water, Potassium Cetyl Phosphate, PEI. (Temperature maintained at 85-90° C.)
3. Slowly added contents of beaker B to beaker A. The gel formation was observed.
4. At 45° C., added Phenoxyethanol.
5. Continued cooling to 25° C.

### Example 9

Foundation Gel Formulation

| Component | Wt % |
|---|---|
| Isododecane | 16.4 |
| Dimethicone | 10 |
| Material from EX 8 | 60 |
| TITANIUM DIOXIDE and | 7.82 |
| DISODIUM | |
| IRON OXIDES and DISODIUM | 1.46 |
| IRON OXIDES and DISODIUM | 0.52 |
| IRON OXIDES and DISODIUM | 0.2 |
| ethylparaben | 0.3 |
| methylparaben | 0.3 |
| glycerin | 3 |
| Total | 100 |

Procedure:
1. In container A, melt material from Example 8 in isododecane and dimethicone until fully dissolved. Bring temp. to 80-90° C.
2. Add iron oxide pigment grind immediately after mixing at high sheer (1000 rpm)
5 In separate container B, mix parabens and glycerin—heat to 80-90° C.
6. Add B to A slowly at high sheer (˜1000 rpm) at 80-90° C.
7. Maintain at 80-90° C. for 30 minutes
8. Turn off heat
9. Cool to RT while mixing at lower shear

### Example 10

Mascara Formulation

| Phase | Component | % wt/wt |
|---|---|---|
| A | Material from EX 8 | 60 |
| B | Iron Oxides | 6.00 |
| B | Isododecane | 9.35 |
| B | Ethylparaben | 0.20 |
| C | DI Water | 9.00 |
| C | Disodium EDTA | 0.10 |
| C | C20-40 Pareth-95 (and) Polyethylene | 8.00 |
| C | Methylparaben | 0.25 |
| C | Pentylene Glycol | 2.00 |
| C | Simethicone | 0.10 |
| D | AQUEOUS POLYURETHANE | |
| | POLYESTER DISPERSION | 5.00 |

1. In a suitable size metal container, add E99999. Heat content to 80° C. or until all uniform.
2. Mix phase B till uniform, add phase B to phase A at around 80° C.
3. Mix phase C till uniform, then add to the mixture of B+A
4. Add Phase D at around 50-60° C.

### Examples Sunscreen Cream

| | Ingredients | EX11 | EX12 | EX13 | EX14 | EX15 | EX16 | EX17 |
|---|---|---|---|---|---|---|---|---|
| Phase | Water | 21.75 | 21.75 | 23.75 | 23.75 | 23.75 | 23.75 | 23.75 |
| A-1 | Methyl Paraben | 0.35 | 0.35 | 0.35 | 0.35 | 0.35 | 0.35 | 0.35 |
| | Ethyl Paraben | 0.20 | 0.20 | 0.20 | 0.20 | 0.20 | 0.20 | 0.20 |
| | Phenoxy Ethanol | 0.70 | 0.70 | 0.70 | 0.70 | 0.70 | 0.70 | 0.70 |
| Phase | PEI-35 | 1.50 | 1.50 | 1.50 | 1.50 | 1.50 | 1.50 | 1.50 |
| A-2 | | | | | | | | |
| Phase B | Isononyl Isonanonoate | 2.00 | 2.00 | 2.00 | 2.00 | 2.00 | 3.00 | 3.00 |
| | Potassium Cetyl Phosphate | 2.20 | 2.20 | 2.20 | 2.20 | 2.20 | 2.20 | 2.20 |
| | BEHENYL ALCOHOL (and) GLYCERYL | 0.00 | 0.00 | 0.00 | 0.00 | 0.00 | 0.50 | 0.00 |
| | STEARATE (and) DISODIUM | | | | | | | |
| | ETHYLENE DICOCAMIDE PEG-15 | | | | | | | |
| | DISULFATE (and) GLYCERYL | | | | | | | |
| | STEARATE CITRATE | | | | | | | |
| | Titanium Dioxide | 10.00 | 7.00 | 10.00 | 10.00 | 6.00 | 0.00 | 6.00 |
| | Octinoxate | 0.00 | 0.00 | 0.00 | 0.00 | 7.50 | 0.00 | 4.00 |
| | Octocrylene | 0.00 | 0.00 | 0.00 | 0.00 | 0.00 | 5.00 | 0.00 |
| | Abvobenzone | 0.00 | 0.00 | 0.00 | 0.00 | 0.00 | 3.00 | 0.00 |
| | BIS-ETHYLHEXYLOXYPHENOL | 0.00 | 0.00 | 0.00 | 0.00 | 0.00 | 0.00 | 2.00 |
| | METHOXYPHENYL TRIAZINE | | | | | | | |
| | Styrene/Acrylates Copolymer | 0.00 | 3.00 | 0.00 | 0.00 | 3.00 | 6.00 | 3.00 |
| | POLY C10-30 ALKYL ACRYLATE | 0.00 | 0.00 | 0.00 | 0.00 | 0.00 | 1.00 | 0.00 |
| | Isododecane | 38.80 | 38.80 | 35.80 | 36.80 | 30.30 | 29.80 | 30.80 |
| | Uncecane (and) Tridecane | 11.00 | 11.00 | 11.00 | 11.00 | 11.00 | 11.00 | 11.00 |
| | Ethylene/Maleic Anhydried/Propylene | 9.00 | 9.00 | 10.00 | 9.00 | 9.00 | 9.00 | 9.00 |
| | Copolymer | | | | | | | |
| | Silica | 2.50 | 2.50 | 2.50 | 2.50 | 2.50 | 3.00 | 2.50 |
| Total | | 100 | 100 | 100 | 100 | 100 | 100 | 100 |

1. Combined Phase A-1 into a beaker. Heated and mixed to 85-90° C.
2. Combined phase B into another beaker. Heated and mixed to 85-90° C. Maintained heated and mixed until homogeneous.
3. Added Phase A2 in to Phase A-1 and mixed for 2-3 minutes.
4. Added phase A-1/A-2 into Phase B. Increased homogenization. Maintained temperature and mixing for 45 minutes.
5. Begin cooling to 50-55° C. temperature.
6. Stopped homogenization and transferred the bulk.

### Example 18

### Sunscreen

| | Ingredients | EX 8 |
|---|---|---|
| Phase | Water | 22.75 |
| A-1 | Methyl Paraben | 0.35 |
| | Ethyl Paraben | 0.20 |
| | Phenoxy Ethanol | 0.70 |
| Phase | TEREPHTHALYLIDENE | 3.00 |
| A-2 | DICAMPHOR SULFONIC | |
| | ACID | |
| | Triethanol Amine | 0.51 |
| Phase | PEI-35 | 1.50 |
| A-3 | | |
| Phase B | Isononyl Isonanonoate | 3.00 |
| | Potassium Cetyl Phosphate | 2.20 |
| | Titanium Dioxide | 6.00 |
| | Octinoxate | 4.00 |
| | DROMETRIZOLE | 1.00 |
| | TRISILOXANE | |
| | ETHYLHEXYL TRIAZONE | 1.00 |
| | Styrene/Acrylates Copolymer | 3.00 |
| | Isododecane | 28.29 |
| | Uncecane (and) Tridecane | 11.00 |
| | Ethylene/Maleic Anhydried/ | 9.00 |
| | Propylene Copolymer | |
| | Silica | 2.50 |
| Total | | 100.00 |

1. Combined Phase A-1 into a beaker. Heated and mixed to 85-90° C.
2. Combined phase B into another beaker. Heated and mixed to 85-90° C. Maintained heated and mixed until homogeneous.
3. Premixed Phase A2 and added into Phase A-1 and mixed.
4. Added Phase A-3 into Phase A1/A2 and mixed for 2-3 minutes.
5. Added phase A-1/A-2/A-3 into Phase B. Increased homogenization. Maintained temperature and mixing for 45 minutes.
6. Began cooling to 50-55° C. temperature.
7. Stopped homogenization and transferred the bulk.

### Examples 19

### Mascara

| Phase | Component | Wt % |
|---|---|---|
| A | Caprylic/capric | 1 |
| | Triglyceride | |
| A | Polylene ethylene | 9.33 |
| | Maleic Anhydride | |
| | Copolymer | |
| A | Iron Oxides | 8 |
| A | Isododecane | 39.92 |
| A | Propylparaben | 0.2 |
| B | DI Water | 34 |
| B | Disodium EDTA | 0.1 |
| B | Potassium Cetyl | 2 |
| | Phosphate | |
| B | Methylparaben | 0.25 |
| B | Pentylene Glycol | 2 |
| B | PEI-35 | 2 |
| C | Simethicone | 0.1 |
| D | Phenoxyethanol (and) | 1.1 |
| | Methylparaben (and) | |
| | Isopropylparaben (and) | |
| | Isobutylparaben (and) | |
| | Butylparaben | |
| | Total | 100 |

Procedures
1. In the main beaker A, Isododecane, Caprylic/capric Triglyceride, Polylene ethylene Maleic Anhydride Copolymer, Propylparaben were added. The contents were heated to 90° C. until all solids have melted.
2. Iron Oxides were added into main beaker and start homogenizing batch for 1 h at 850 RPM. (Temperature maintained at 85-90° C.)
3. In a side beaker B, ID water, Disodium EDTA, Potassium Cetyl Phosphate, Methylparaben, Pentylene Glycol were added and mixed until uniform, and heated to 90° C.
4. In the side beaker B, PEI was added and mixed until PEI dissolved. (Temperature maintained at 85-90° C.)
5. Side beaker B was slowly added to main beaker A. Then Simethicone was added to the mixture. The gel formation was observed in 5 minutes after mixing A and B.
6. During the gel formation, the mixing speed was slowed from 250 RPM to 100 RPM to 50 RPM.
7. Once the gel network became thick enough, a sweep blade was used. Cooling was started using 50 RPM.
8. At 35° C., the mixture of Phenoxyethanol (and) Methylparaben (and) Isopropylparaben (and) Isobutylparaben (and) Butylparaben was added.
9. Continued cooling to 25° C. and dropped batch at 25° C.

### Example 20

### Lipstick

| Phase | Ingredient | % wt/wt |
|---|---|---|
| A | Octododecanol | 4.20 |
| A | Octyldodecyl Neopantanoate | 19.30 |
| A | Hydrogenated Polydecene | Q.S. |
| A | Polypropylene-ethylene-maleic | 10.00 |
| | acid anhydride copolymer wax | |
| A | Polyethylene | 8.00 |
| A | VP/Eicosene Copolymer | 3.00 |
| A | Color Pigments | 2.77 |
| B | Deionized Water | 15.00 |
| B | PEI-35 | 0.25 |
| B | Glycerin | 3.00 |
| | Total | 100.00 |

Procedure:
1. The following were added to a suitable size beaker A and heated to 95 Celsius degrees: octododecanol, octyldodecyl neopantanoate, hydrogenated polydecene, polyethylene, VP/Eicosene Copolymer, Color pigment, Polypropylene-ethylene-maleic acid anhydride copolymer wax.
2. When enough solids had melted, the contents were mixed with moderate speed until all solids had melted at 95 Celsius degrees.
3. The temperature was slightly lowered to 85 Celsius degrees and pigments and mica were added.
4. The contents of main beaker A was transferred to a Silverson mixer for emulsification while maintaining the temperature at 85-90 Celsius degrees.
5. In a separate beaker 2, glycerin and PEI-35 were added into DI water and mixed and heated to 85 Celsius degrees.
6. The contents of side beaker B was added dropwise into the beaker A while emulsifying at 9000 rpm under the Silverson mixer for 30 minutes.
7. Afterward, the emulsification speed was lowered to 2000 rpm for 5 minutes.
8. The contents were poured into lipstick molds at 80 Celsius degrees.
9. The lipstick in molds was placed in a cooling tunnel for 15 minutes at −10 Celsius degrees. Once cooled, the lipstick in molds were removed from the cooling tunnel to equilibrate to 25 Celsius degrees and removed from mold after lipsticks had thawed to 25 Celsius degrees.

### Example 21

### Foundation

| | | Weight |
|---|---|---|
| Phase | INCL Name | % |
| A | isododecane | Q.S. |
| A | Polypropylene-ethylene-maleic | 9 |
| | acid anhydride copolymer wax | |
| A | Sunsphere Silica | 2.5 |
| A | Potassium Cetyl Phosphate | 2.2 |
| A | Pigments | 8 |
| B | DI Water | 22.25 |
| B | PEI-35 | 1.5 |
| B | salt | 0.5 |
| B | DISODIUM EDTA | 0.20 |
| B | glycerin | 2.75 |
| B | propylene glycol | 2.00 |
| B | PHENOXY-2 ETHANOL | 0.50 |
| B | methylparaben | 0.20 |
| B | CHLORPHENESIN | 0.20 |
| B | ETHYL PARABEN | 0.2 |
| | Total | 100 |

Procedure
1. In container A, Polypropylene-ethylene-maleic acid anhydride copolymer wax was melted in the isododecane until fully dissolved. The temperature was brought to 90° C.
2. While maintaining the temperature, Potassium Cetyl Phosphate, sunsphere silica and pigments were added to container A until fully dissolved.
3. In a separate container B, PEI-35, water, glycerin, salt, Disodium EDTA, propylene glycol and preservatives were mixed and heated to 90° C.
4. The contents of container B were added to the contents of container A slowly at high sheer (1000 rpm).
5. Heat was maintained at 70° C.-80° C. for 20 minutes while maintaining high sheer mixing.
6. The mixture was cooled to room temperature while mixing.

What is claimed is:

1. A water-insoluble reaction product comprising at least one oil-soluble polar modified polymer and at least one polyamine.

2. The reaction product of claim 1, wherein the oil-soluble polar modified polymer is a C₂-C₃ polar modified wax.

3. The reaction product of claim 2, wherein the C₂-C₃ polar modified wax comprises maleic anhydride units.

4. The reaction product of claim 3, wherein the polyamine is a polyethyleneimine.

5. A composition comprising a water-insoluble reaction product comprising at least one oil-soluble polar modified polymer and at least one polyamine.

6. The composition of claim 5, wherein the oil-soluble polar modified polymer is a C₂-C₃ polar modified wax.

7. The composition of claim 6, wherein the C₂-C₃ polar modified wax comprises maleic anhydride units.

8. The composition of claim 7, wherein the polyamine is a polyethyleneimine.

9. The composition of claim 5, wherein the composition is an emulsion.

10. The composition of claim 9, further comprising at least one colorant.

11. The reaction product of claim 1, wherein the at least one oil-soluble polar modified polymer consists of polypropylene, polyetheylene and maleic anhydride units.

12. A composition comprising the reaction product of claim 4.

13. The composition of claim 5, further comprising water.

14. The composition of claim 13, wherein the composition comprises water in an amount ranging from 5% to 50% by weight with respect to the weight of the composition.

15. A water-insoluble reaction product produced by combining an aqueous phase comprising at least one polyamine and an oil phase comprising at least one oil-soluble polar modified polymer to form the reaction product.

16. The composition of claim 5, wherein the composition is in the form of a gel.
